(19) United States
(12) Patent Application Publication
(10) Pub. No.: US 2004/0152661 A1
(43) Pub. Date: Aug. 5, 2004

(54) Methods and compositions for treating viral diseases

(75) Inventor: Joseph Armstrong Martin, Menlo Park, CA (US)

Correspondence Address: ROCHE PALO ALTO LLC, PATENT LAW DEPT. M/S A2-250, 3431 HILLVIEW AVENUE, PALO ALTO, CA, 94304, US

(73) Assignee: Syntex (U.S.A.) LLC

(21) Appl. No.: 10/753,840

(22) Filed: Jan. 8, 2004

Related U.S. Application Data
(60) Provisional application No. 60/438,878, filed on Jan. 9, 2003.

(51) Int. Cl. A61K 31/7072

(52) U.S. Cl. 514/50

(57) ABSTRACT

The present invention provides methods of preventing or treating West Nile virus as well as infections caused by other viruses of the Flaviviridae family in animals comprising administering to the animal a tehrapeutically effective amount of a compound of formula I either as alone or in combination with immunomodulators.

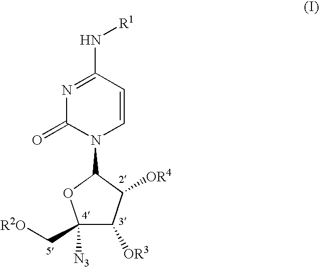

### CROSS REFERENCE TO PRIOR APPLICATION

This application claims benefit under Title 35 U.S.C. 119(e) of U.S. Provisional Application No. 60/438,878, filed Jan. 9, 2003, which is hereby incorporated by reference in its entirety.

### FIELD OF THE INVENTION

The invention relates to the field of antiviral therapy and in particular to nucleoside derivatives for diseases mediated by viruses of the family Flaviviridae. The invention provides methods for treatment or prophylaxis of diseases by viruses of the family Flaviviridae and pharmaceutical compositions for treatment or prophylaxis of diseases by viruses of the family Flaviviridae.

### BACKGROUND

The flaviviridae comprise over 60 viruses. Arthropod vectors including ticks and mosquitoes disseminate many virus of this family. Twenty-six flaviviridae are known to produce human disease. Among the common serious human diseases caused by this family are: dengue fever, west Nile virus, St. Louis encephalitis, Japanese encephalitis, and yellow fever. Human hepatitis C virus HCV is mediated by a virus which has similarities to the flaviviruses and the animal pestiviruses. Although it is a member of the Flavivirus genus of, there is 45-49% homology with the pestiviruses sequence in the 5′-untranslated region while the hydrophobicity profile of the HCV polyprotein is more closely reminiscent of the flaviviruses.

West Nile virus an arthropod-borne flavivirus, has emerged in recent years as a deadly health threat to not only humans, but also to other animal species such as horses and birds. New York was the first area in North American to report cases of West Nile virus infections. West Nile virus infection in humans has been found previously only in Africa, the Middle East and Eastern Europe.

Among infected humans, approximately one in every 150 to 300 become ill with fever, myalgia and possible rash. Among those who are symptomatic, approximately 10-15% will have evidence of meningitis (headache, stiff neck) or encephalitis (change of mental status, peripheral neurologic abnormalities, muscle weakness). However, almost all fatalities have occurred among humans over the age of 50. The fatality rate among patients with central nervous system infection was and 11% in New York. Fatalities have been due to prolonged central nervous system dysfunction requiring ventilatory support and leading to secondary complications. Prolonged neurologic symptoms have occurred in survivors of encephalitis.

Denque fever is endemic to the tropics, especially the Caribbean, the Pacific and some areas of West Africa. Denque is endemic in tropical areas where Stegomyia species are constantly active. Aedes aegypti is probably the most common vector in urban areas. Dengue fever typically is manifested by a sudden onset of fever which often becomes biphasic, severe headache, pain behind the eyes, backache, chilliness and generalized muscle and joint pains. Severe manifestations of dengue fever include dengue hemorrhagic fever which can lead to plasma leakage from the vascular system and severe shock which has a poor prognosis.

Treatment of dengue fever typically is supportive with fluid replacement therapy when appropriate. Mortality in simple cases of dengue fever is low; however convalescence can be last for weeks. Mortality in dengue hemorrhagic fever ranges from 6-30% and infants are most susceptible.

The Japanese encephalitis antigenic complex includes Japanese encephalitis, West Nile Virus, Saint Louis encephalitis and Murray Valley encephalitis. These viruses exhibit approximately 60% sequence homology.

Saint Louis Encephalitis is one of the most common arbovirus transmitted diseases in the US and has caused major epidemics. The disease is most common in the Mississippi-Ohio river basin, Texas and Florida. The disease is transmitted by Culex spp. and can appear whenever standing water produce optimal conditions for mosquito breeding. Common clinical symptoms include encephalitis, aseptic meningitis and febrile headache. The fatality rate ranges from 2% in the young to about 22% in the elderly. Treatment is symptomatic and convalescence can be prolonged. Japanese encephalitis is occurs throughout the eastern seaboard of Asia and through parts of India and Sri Lanka. It is spread by rice-field breeding Culex spp. and the virus is amplified in swine. Initial symptoms are similar to those of St. Louis encephalitis. Treatment is supportive and convalescence is often protracted and sequelae are common in children. Case fatality has ranged from 20% to 70% during epidemics but the high rate is probably indicative of a lack of quality medical care. Murray Valley encephalitis occurs primarily in Australia although scattered cases have been reported in New Guinea. The disease is transmitted by Culex spp. and birds and mammals appear to reservoirs for infection. The disease begins with headache, fever and generalized malaise which become progressively more severe.

The incidence of yellow fever has been reduced by the existence of an effective vaccine. Outbreaks of yellow fever are still common in South American and Asian countries and it is an important cause of viral hemorrhagic fever. The disease is transmitted by Aedes spp. Mortality is low but treatment of the disease is symptomatic.

These arboviral diseases have no effective treatment once an outbreak occurs. Thus there exists an urgent need for new therapies to ameliorate the effect of these arboviral disease. West Nile virus also effects various animal species including horses and birds and therapy to control arboviral outbreaks in animals also is desirable.

Therapies for most viral diseases are still relatively limited and in the absence of a prophylactic vaccine most therapy is symptomatic. Ribavirin and interferon alpha-2b are active against hepatitis C virus which is a member of the genus Flavivirus. West Nile virus is also a member of the genus Flavivirus. U.S. patent application Ser. No. 2002/0061290 A1 (J. J. Rahal) describe the use of ribavirin, interferon-2b or combinations thereof for treatment of West Nile Virus and other Flavivirus-induced diseases.

Although ribavirin is used in the treatment of viral diseases, it unfortunately is has side effects which can limit its potential use. The major toxicity of ribavirin is hemolysis due to accumulation of ribavirin triphosphate within erythrocytes leading to a decreased life span. Such accumulation occurs due to inability of erythrocytes to dephosphorylate the triphosphate. Inhibition of erythrocyte release from bone marrow occurs at high doses (30 mg/kg). Hemolysis is related to the dose and duration of therapy, and is reversible after discontinuation. Ribavirin is teratogenic and should not be given during, or within 6 months of pregnancy. Bioavailability is increased in patients with renal dysfunction. Nucleoside derivatives frequently exhibit high levels of biological activity; however, their practical utility is often limited by suboptimal physical properties and poor pharmacokinetics and the present invention relates also to nucleoside prodrugs with pharmacokinetic properties.

Thus a continuing need for to identify new antiviral therapies with high efficacy and reduced side effects.

### DETAILED DESCRIPTION OF THE INVENTION

Surprisingly a series of nucleic acid derivatives have now been identified which are efficacious against viruses in the family Flaviviridae. The utility of these compounds against Hepatitis C virus has been disclosed in U.S. patent application Ser. No. 10/167,106, filed Jun. 11, 2002 which claims priority the United Kingdom application GB 0114286.8 which was filed Jun. 12, 2001. Prodrugs of an antiviral nucleoside have also been described in U.S. patent application Ser. No. 60/427,447 filed Nov. 19, 2002. Both U.S. patent application Ser. Nos. 10/167,106 and 60/427,447 are hereby incorporated in their entirety by reference.

The present invention relates to methods of treating viral infections mediated by a virus of family Flaviviridae by administering to a animal in need thereof a therapeutically effective amount of a compound according to formula I

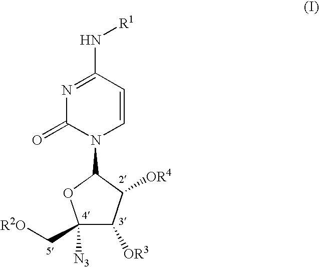

wherein:

R¹ and R² are independently selected from the group consisting of hydrogen, COR⁵, CO₂R⁵ and COCH(R⁶)NHR⁷;

R³ and R⁴ independently of the other are selected from the group consisting of hydrogen, COR⁵, CO₂R⁵ and COCH(R⁶)NHR⁷, or R³ and R⁴ taken together are selected from the group consisting of CH₂, C(CH₃)₂ and CHPh;

R⁵ is independently selected from the group consisting of C₁₋₆ unbranched or branched alkyl, C₁₋₆ unbranched or branched alkenyl, C₁₋₆ unbranched or branched alkynyl, C₁₋₆ lower haloalkyl, C₃₋₈ cycloalkyl, alkyl substituted C₃₋₈ cycloalkyl, phenyl optionally independently substituted with one to three substituents selected from the group consisting of halo, lower alkyl, lower alkoxy, lower thioalkyl, lower alkyl sulfinyl, lower alkyl sulfonyl, nitro, and cyano, CH₂Ph wherein in phenyl ring is optionally substituted as described above and CH₂OPh wherein in phenyl ring is optionally substituted as described above;

R⁶ is selected from the group consisting of the side chains of naturally occurring amino acids and C₁₋₅ unbranched or branched alkyl;

R⁷is selected from the group consisting of hydrogen, R⁵OCO, and;

hydrates, solvates, clathrates and acid addition salts thereof; with the proviso that the viral disease is not mediated by Hepatitis C Virus; and, pharmaceutical compositions comprising such compounds or for the preparation of medicaments for such treatment.

In one embodiment of the invention there is provided a method to treat viral infections mediated by a virus of family Flaviviridae by administering to a animal in need thereof a therapeutically effective amount of a compound according to formula I wherein R¹, R², R³ and R⁴ are as defined hereinabove.

In another embodiment of the present invention there is provided a method to treat viral infections mediated by dengue fever virus, West Nile virus, St. Louis encephalitis, Japanese encephalitis or Murray Valley encephalitis by administering to an animal in need thereof a therapeutically effective amount of a compound according to formula I wherein R¹, R², R³ and R⁴ are as defined hereinabove.

In another embodiment of the present invention there is provided a method to treat viral infections mediated by a virus of family Flaviviridae by administering to an animal in need thereof a therapeutically effective amount of a compound according to formula I wherein R¹, R², R³ and R⁴ are hydrogen.

In another embodiment of the present invention there is provided a method to treat viral infections mediated by a virus of family Flaviviridae wherein said viral infections are mediated by dengue fever virus, West Nile virus, St. Louis encephalitis, Japanese encephalitis or Murray Valley encephalitis by administering to an animal in need thereof a therapeutically effective amount of a compound according to formula I wherein R¹, R², R³ and R⁴ are hydrogen.

In one embodiment of the present invention there is provided a method to treat viral infections mediated by a virus of family Flaviviridae by administering to a animal in need thereof a therapeutically effective amount of a compound according to formula I wherein R¹, R², R³ and R⁴ are as defined hereinabove and the compound is delivered in a dose of between 1 and 100 mg/kg of body weight of the patient per day.

In one embodiment of the present invention there is provided a method to treat viral infections mediated by a virus of family Flaviviridae by administering to a human in need thereof a therapeutically effective amount of a compound according to formula I wherein R¹, R², R³ and R⁴ are as defined hereinabove.

In one embodiment of the present invention there is provided a method to treat viral infections mediated by a virus of family Flaviviridae by co-administering to an animal in need thereof a therapeutically effective amount of a compound according to formula I wherein R¹, R², R³ and R⁴ and an immune system modulator.

In one embodiment of the present invention there is provided a method to treat viral infections mediated by a virus of family Flaviviridae by co-administering to an animal in need thereof a therapeutically effective amount of a compound according to formula I wherein R¹, R², R³ and R⁴ are as defined hereinabove, and an interferon, interleukin, tumor necrosis factor, colony stimulating factor, or antiviral agent.

In one embodiment of the present invention there is provided a method to treat viral infections mediated by a virus of family Flaviviridae by co-administering to an animal in need thereof a therapeutically effective amount of a compound according to formula I wherein R¹, R², R³ and R⁴ and an interferon or chemically derivatized interferon.

In one embodiment of the present invention there is provided a method to treat viral infections mediated by a virus of family Flaviviridae by co-administering to an animal in need thereof a therapeutically effective amount of a compound according to formula I wherein R¹, R², R³ and R⁴ are as defined hereinabove and an interferon-a or chemically derivatized interferon-a.

In one embodiment of the present invention there is provided a pharmaceutical composition for treating a viral infections mediated by a virus of family Flaviviridae comprising a therapeutically effective quantity of a compound of formula I

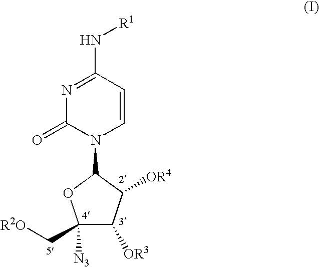

wherein:

R¹ and R² are independently selected from the group consisting of hydrogen, COR⁵, CO₂R⁵ and COCH(R⁶)NHR⁷;

R³ and R⁴ independently of the other are selected from the group consisting of hydrogen, COR⁵, CO₂R⁵ and COCH(R⁶)NHR⁷, or R³ and R⁴ taken together are selected from the group consisting of CH₂, C(CH₃)₂ and CHPh;

R⁵ is independently selected from the group consisting of C₁₋₆ unbranched or branched alkyl, C₁₋₆ unbranched or branched alkenyl, C₁₋₆ unbranched or branched alkynyl, C₁₋₆ lower haloalkyl, C₃₋₈ cycloalkyl, alkyl substituted C₃₋₈ cycloalkyl, phenyl optionally independently substituted with one to three substituents selected from the group consisting of halo, lower alkyl, lower alkoxy, lower thioalkyl, lower alkyl sulfinyl, lower alkyl sulfonyl, nitro, and cyano, CH₂Ph wherein in phenyl ring is optionally substituted as described above and CH₂OPh wherein in phenyl ring is optionally substituted as described above;

R⁶ is selected from the group consisting of the side chains of naturally occurring amino acids and C₁₋₅ unbranched or branched alkyl;

R⁷ is selected from the group consisting of hydrogen, R⁵OCO, and;

hydrates, solvates, clathrates and acid addition salts thereof; and, in combination with one or more pharmaceutically acceptable carriers and excipients; pharmaceutical compositions comprising such compounds; or, for the preparation of medicaments for such treatment;

with the proviso that the viral infection is not mediated by Hepatitis C Virus.

Compounds of the present invention include pro-drugs or bioprecursors of the parent nucleoside and are converted in vivo to the compound of formula I wherein R¹, R², R³, and R⁴ are hydrogen. Pro-drug derivatives include carboxylic esters in which the non-carbonyl moiety of the ester group is selected from unbranched or branched alkyl (e.g. methyl, n-propyl, n-butyl or t-butyl), alkoxyalkyl (e.g. methoxymethyl), aralkyl (e.g. benzyl), aryloxyalkyl (e.g. phenoxymethyl), aryl (e.g. phenyl optionally substituted by halogen, C₁₋₄ alkyl or C₁₋₄ alkoxy or amino); amino acid esters (e.g. L-valyl or L-isoleucyl) or pharmaceutically acceptable salts thereof. The preparation is carried out according to known methods in the art, for example methods known from textbooks on organic chemistry (e.g. from J. March (1992), “Advanced Organic Chemistry: Reactions, Mechanisms, and Structure”, 4th ed. John Wiley & Sons).

### DEFINITIONS

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined hereinabove” refers to the first definition provided in the Detailed Description of the Invention.

The terms “optional” or “optionally” as used herein means that a described event or circumstance may or may not occur, and that the description includes instances where said event or circumstance occurs and instances in which it does not. For example, “optionally substituted phenyl” means that the phenyl may or may not be substituted and that the description includes both unsubstituted phenyl and phenyl wherein there is substitution.

Compounds of the present invention may have asymmetric centers located on the side chain of a carboxylic ester, amide or carbonate moiety that produce diastereomers when linked to the nucleoside. All stereoisomers on the side chain of the compounds of the instant invention are contemplated, either in admixture or in pure or substantially pure form. The definition of the compounds according to the invention embraces all possible stereoisomers and their mixtures. It also embraces the racemic forms as well as the isolated optical isomers. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

All configurational isomers of compounds of the present invention are contemplated, either in admixture or in pure or substantially pure form. The definition of compounds of the present invention embraces both cis and trans isomers of cycloalkyl rings.

The term “alkyl” as used herein denotes a unbranched or branched chain hydrocarbon residue containing 1 to 12 carbon atoms. The term “lower alkyl” denotes a unbranched or branched chain hydrocarbon residue containing 1 to 6 carbon atoms. Representative lower alkyl groups include methyl, ethyl, propyl, i-propyl, n-butyl, i-butyl, t-butyl or pentyl.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl.

The term “alkenyl” as used herein denotes an unsubstituted [or substituted] hydrocarbon chain radical having from 2 to 7 carbon atoms, preferably from 2 to 4 carbon atoms, and having one or two olefinic double bonds, preferably one olefinic double bond. Examples are vinyl, 1-propenyl, 2-propenyl (allyl) or 2-butenyl (crotyl).

The term “alkynyl” as used herein denotes an unsubstituted hydrocarbon chain radical having from 2 to 7 carbon atoms, [preferably 2 to 4 carbon atoms], and having one or where possible two triple bonds, [preferably one triple bond]. Examples are ethynyl, 1-propynyl, 2-propynyl, 1-butynyl, 2-butynyl or 3-butynyl.

The term “alkoxy” as used herein denotes an unsubstituted unbranched or branched chain alkyloxy group, -O(alkyl), wherein the “alkyl” portion is as defined above such as methoxy, ethoxy, n-propyloxy, i-propyloxy, n-butyloxy, i-butyloxy, t-butyloxy, pentyloxy, hexyloxy, heptyloxy including their isomers. “Lower alkoxy” as used herein denotes an alkoxy group with a “lower alkyl” group as previously defined.

The term “alkylthio” as used herein denotes a unbranched or branched chain (alkyl)S- group wherein the “alkyl” portion is as defined above. Examples are methylthio, ethylthio, n-propylthio, i-propylthio, n-butylthio, i-butylthio or t-butylthio.

The term “alkoxyalkyl” as used herein denotes an alkoxy group as defined above which is bonded to an alkyl group as defined above. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, t-butyloxybutyl, methoxypentyl, ethoxypentyl, and propyloxypentyl including their isomers.

The term “hydroxyalkyl” as used herein denotes a unbranched or branched chain alkyl group as defined above wherein 1, 2, 3 or more hydrogen atoms are substituted by a hydroxy group. Examples are hydroxymethyl, 1-hydroxyethyl, 2-hydroxyethyl, 1-hydroxypropyl, 2-hydroxypropyl, 3-hydroxypropyl, hydroxyisopropyl, hydroxybutyl and the like.

The term “aryl” as used herein denotes an optionally substituted monocyclic or polycyclic-aromatic group comprising carbon and hydrogen atoms. Examples of suitable aryl groups include, but are not limited to, phenyl and naphthyl (e. g. 1-naphthyl or 2-naphthyl). Suitable substituents for aryl are selected from the group consisting of alkyl, alkenyl, alkynyl, aryloxy, cycloalkyl, acyl, acylamino, alkoxy, amino, alkylamino, dialkylamino, halogen, haloalkyl, hydroxy, nitro and cyano.

The term “acyl” (“alkylcarbonyl”) as used herein denotes a group of formula C(═O)R wherein R is hydrogen, unbranched or branched alkyl containing 1 to 7 carbon atoms or a phenyl group. Most preferred acyl groups are those wherein R is hydrogen, an branched or unbranched alkyl chain or containing 1 to 6 carbon atoms or an optionally substituted phenyl group.

The term halogen stands for fluorine, chlorine, bromine or iodine, preferably fluorine, chlorine, bromine.

The term “amino acid” as used herein refers to naturally occurring amino acids, as well as to optical isomers (enantiomers and diastereomers), synthetic analogs and derivatives thereof. α-Amino acids comprise a carbon atom bonded to a carboxyl group, an amino group, a hydrogen atom and a unique “side chain” group. The side chains of naturally occurring amino acids are well known and include hydrogen, alkyl, hydroxyalkyl, thioalkyl, alkylthioalkyl, branched alkyl, carboxyalkyl, carboxamidoalkyl, aminoalkyl, arylalkyl, and heteroarylalkyl moieties. The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine.

The term “chemically-derivatized interferon” as used herein refers to an interferon molecule covalently linked to a polymer which alters the physical and/or pharmacokinetic properties of the interferon. A non-limiting list of such polymers include polyalkylene oxide homopolymers such as polyethylene glycol (PEG) or polypropylene glycol (PPG), polyoxyethylenated polyols, copolymers thereof and block copolymers thereof, provided that the water solubility of the block copolymers is maintained. One skilled in the art will be aware of numerous approaches to linking the polymer and interferon (for example, see A. Kozlowski and J. M. Harris J. Control. Release 2001 72(1-3):217-24). A non-limiting list of chemically derivatized IFNα contemplated in the present patent includes peginterferon-α-2a (PEGASYS®) and peginterferon-α-2b (PEGINTRON®).

Compounds of formula I which are basic can form pharmaceutically acceptable salts with inorganic acids such as hydrohalic acids (e.g. hydrochloric acid and hydrobromic acid), sulphuric acid, nitric acid and phosphoric acid, and the like, and with organic acids (e.g. with acetic acid, tartaric acid, succinic acid, fumaric acid, maleic acid, malic acid, salicylic acid, citric acid, methanesulphonic acid and p-toluene sulphonic acid, and the like). The formation and isolation of such salts can be carried out according to methods known in the art.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “hydrate” as used herein means a compound of the invention or a salt thereof, that further includes a stoichiometric or non-stoichiometric amount of water bound by non-covalent internlolecular forces.

The term “clathrate” as used herein means a compound of the invention or a salt thereof in the form of a crystal lattice that contains spaces (e.g., channels) that have a guest molecule (e.g.), a solvent or water) trapped within.

### DOSAGE AND ADMINISTRATION

While nucleoside derivatives of the present invention are optimized for delivery across the gastrointestinal mucosa, these compounds can be efficacious when administered by other routes of administration including continuous (intravenous drip) topical parenteral, intramuscular, intravenous, subcutaneous, transdermal (which may include a penetration enhancement agent), buccal, nasal and suppository administration, among other routes of administration. Oral administration can be in the form of tablets, coated tablets, dragées, hard and soft gelatine capsules, solutions, emulsions, syrups, or suspensions

For the manufacture of pharmaceutical preparations, the nucleoside derivatives, as well as their pharmaceutically useable salts, can be formulated with a therapeutically inert, inorganic or organic excipient for the production of tablets, coated tablets, dragées, hard and soft gelatine capsules, solutions, emulsions or suspensions. The compounds of formula I can be formulated in admixture with a pharmaceutically acceptable carrier. For example, the compounds of the present invention can be administered orally as pharmacologically acceptable salts. Because the compounds of the present invention are mostly water soluble, they can be administered intravenously in physiological saline solution (e.g., buffered to a pH of about 7.2 to 7.5). Conventional buffers such as phosphates, bicarbonates or citrates can be used in the present compositions. Suitable excipients for tablets, coated tablets, dragées, and hard gelatin capsules are, for example, lactose, corn starch and derivatives thereof, talc, and stearic acid or its salts. If desired, the tablets or capsules may be enteric-coated or sustained release by standard techniques. Suitable excipients for soft gelatine capsules are, for example, vegetable oils, waxes, fats, semi-solid and liquid polyols. Suitable excipients for injection solutions are, for example, water, saline, alcohols, polyols, glycerine or vegetable oils. Suitable excipients for suppositories are, for example, natural and hardened oils, waxes, fats, semi-liquid or liquid polyols. Suitable excipients for solutions and syrups for enteral use are, for example, water, polyols, saccharose, invert sugar and glucose. T he pharmaceutical preparations can also contain preservatives, solubilizers, stabilizers, wetting agents, emulsifiers, sweeteners, colorants, flavorants, salts for adjustment of the osmotic pressure, buffers, masking agents or antioxidants. The pharmaceutical preparations may also contain other therapeutically active agents known in the art.

Other suitable pharmaceutical carriers and their formulations are described in Remington: The Science and Practice of Pharmacy 1995, edited by E. W. Martin, Mack Publishing Company, 19th edition, Easton, Pennsylvania. Representative pharmaceutical formulations containing a compound of the present invention are described in Examples 6-8. A skilled formulation scientist may modify the formulations within the teachings of the specification to provide numerous formulations for a particular route of administration without rendering the compositions of the present invention unstable or compromising their therapeutic activity.

In particular, the modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. That dosage can vary within wide limits and will, of course, be adjusted to the individual requirements in each particular case. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 100 mg/kg body weight per day. A typical preparation will contain from about 5% to about 95% active compound (w/w). The daily dosage can be administered as a single dosage or in divided dosages, typically between 1 and 5 dosages per day.

The term “animals” as used herein include mammals, e.g., humans, companion animals e.g., dogs and cats, laboratory animals, e.g., rats and mice, and farm animals, e.g., swine, horses and cows. Animals include agronomically important avian species e.g., chickens. ducks and turkeys, companion and wild avian species.

In another embodiment of the invention, the active compound or a salt can be administered in combination with another antiviral agent, such as an anti-hepatitis agent. When the active compound or its derivative or salt are administered in combination with another antiviral agent the activity may be increased over the parent compound. This can easily be assessed by preparing the derivative and testing its anti-HCV activity according to the method described herein.

It will be understood that references herein to treatment extend to prophylaxis as well as to the treatment of existing conditions, and that the treatment of animals includes the treatment of humans as well as other animals. Furthermore, treatment of a HCV infection, as used herein, also includes treatment or prophylaxis of a disease or a condition associated with or mediated by HCV infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The nucleoside derivatives or the medicaments thereof may be used in monotherapy or combination therapy, i.e. the treatment may be in conjunction with the administration of one or more additional therapeutically active substance(s), for example, an immune system modulator such as an interferon, interleukin, tumor necrosis factor or colony stimulating factor; an antiviral agent or an anti-inflammatory agent. When the treatment is combination therapy, such administration may be concurrent or sequential with respect to that of the nucleoside derivatives. Concurrent administration, as used herein thus includes administration of the agents at the same time or at different times.

It will be understood that references herein to treatment extend to prophylaxis as well as to the treatment of existing conditions, and that the treatment of animals includes the treatment of humans as well as other animals. Furthermore, treatment of a Hepatitis C Virus (HCV) infection, as used herein, also includes treatment or prophylaxis of a disease or a condition associated with or mediated by Hepatitis C Virus (HCV) infection, or the clinical symptoms thereof.

### EXAMPLE 1

Antiviral Assays

Antiviral assays were run using the procedure of Sidwell and Hoffman (Appl Microbiol. 1971 22:797-801). These assays were previously utilized to establish the antiviral activity of ribavirin. (R. W. Sidwell et al. Science 1972 177:705-706; Sidwell et al. Antimicrob. Ag. Chemother. 1973 3:235-241).

### EXAMPLE 2

Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### EXAMPLE 3

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

### EXAMPLE 4

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

### EXAMPLE 5

Composition for Parenteral Administration

| Ingredient | % wt./wt. | |
|---|---|---|
| Active ingredient | 0.25 | g |
| Sodium Chloride | q.s. to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### EXAMPLE 6

Composition for Suppository

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### EXAMPLE 7

Topical Formulation

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, with reference to the specific embodiments for purposes of clarity and understanding. It will be obvious to one of skill in the art that changes and modifications may be made and equivalents substituted without departing from the true spirit and scope of the invention. Therefore, it is to be understood that the above description is intended to be illustrative and not restrictive. Many modifications may be made to adapt a particular situation, material, composition of matter, process, or process step or steps, to the objective spirit and scope of the present invention. All such modifications are intended to be within the scope of the claims appended hereto

All patents, patent applications and publications cited in this application are hereby incorporated by reference in their entirety for all purposes to the same extent as if each individual patent, patent application or publication were so individually denoted.

We claim:

1. A method of treating a viral infection mediated by a virus of family Flaviviridae by administering to an animal in need thereof a therapeutically effective amount of a compound according to formula I 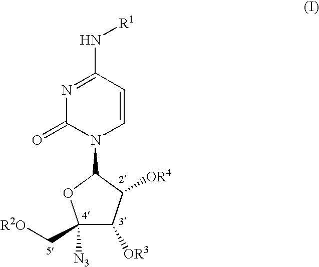
wherein:
R¹ and R² are independently selected from the group consisting of hydrogen, COR⁵, CO₂R⁵ and COCH(R⁶)NHR⁷;
R³ and R⁴ independently of the other are selected from the group consisting of hydrogen, COR⁵, CO₂R⁵ and COCH(R⁶)NHR⁷, or R³ and R⁴ taken together are selected from the group consisting of CH₂, C(CH₃)₂ and CHPh;
R⁵ is independently selected from the group consisting of C₁₋₆ unbranched or branched alkyl, C₁₋₆ unbranched or branched alkenyl, C₁₋₆ unbranched or branched alkynyl, C₁₋₆ lower haloalkyl, C₃₋₈ cycloalkyl, alkyl substituted C₃₋₈ cycloalkyl, phenyl optionally independently substituted with one to three substituents selected from the group consisting of halo, lower alkyl, lower alkoxy, lower thioalkyl, lower alkyl sulfinyl, lower alkyl sulfonyl, nitro, and cyano, CH₂Ph wherein in phenyl ring is optionally substituted as described above and CH₂OPh wherein in phenyl ring is optionally substituted as described above;
R⁶ is selected from the group consisting of the side chains of naturally occurring amino acids and C₁₋₅ unbranched or branched alkyl;
R⁷ is selected from the group consisting of hydrogen, R⁵OCO, and;
hydrates, solvates, clathrates and acid addition salts thereof; and, pharmaceutical compositions comprising such compounds or for the preparation of medicaments for such treatment;
with the proviso that the viral infection is not mediated by Hepatitis C Virus.

2. A method according to claim 1 wherein said viral infections are mediated by dengue fever virus, West Nile virus, St. Louis encephalitis virus, Japanese encephalitis virus or Murray Valley encephalitis virus.

3. A method according to claim 1 wherein R¹, R², R³ and R⁴ are hydrogen.

4. A method according to claim 1 wherein said viral infections are mediated by dengue fever virus, West Nile virus, St. Louis encephalitis virus, Japanese encephalitis virus or Murray Valley encephalitis virus.

5. The method of claim 4 wherein the compound is delivered in a dose of between 1 and 100 mg/kg of body weight of the patient per day.

6. The method of claim 1 wherein the animal is a human.

7. The method of claim 1 further comprising co-administering an immune system modulator.

8. The method of claim 7 wherein the immune system modulator is an interferon, interleukin, tumor necrosis factor or colony stimulating factor, an antiviral agent or an anti-inflammatory agent.

9. The method of claim 8 wherein the immune system modulator is an interferon or chemically derivatized interferon.

10. The method of claim 9 wherein the immune system modulator is interferon-α or chemically derivatized interferon-a.

11. A pharmaceutical composition for treating a viral infection mediated by a virus of family Flaviviridae comprising a therapeutically effective quantity of a compound of formula I 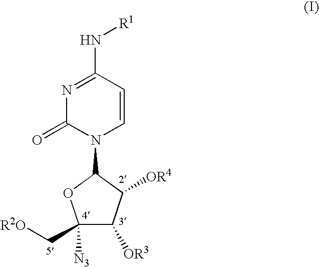
wherein:
R¹ and R² are independently selected from the group consisting of hydrogen, COR⁵, CO₂R⁵ and COCH(R⁶)NHR⁷;
R³ and R⁴ independently of the other are selected from the group consisting of hydrogen, COR⁵, CO₂R⁵ and COCH(R⁶)NHR⁷, or R³ and R⁴ taken together are selected from the group consisting of CH₂, C(CH₃)₂ and CHPh;
R⁵ is independently selected from the group consisting of C₁₋₆ unbranched or branched alkyl, C₁₋₆ unbranched or branched alkenyl, C₁₋₆ unbranched or branched alkynyl, C₁₋₆ lower haloalkyl, C₃₋₈ cycloalkyl, alkyl substituted C₃₋₈ cycloalkyl, phenyl optionally independently substituted with one to three substituents selected from the group consisting of halo, lower alkyl, lower alkoxy, lower thioalkyl, lower alkyl sulfinyl, lower alkyl sulfonyl, nitro, and cyano, CH₂Ph wherein in phenyl ring is optionally substituted as described above and CH₂OPh wherein in phenyl ring is optionally substituted as described above;
R⁶ is selected from the group consisting of the side chains of naturally occurring amino acids and C₁₋₅ unbranched or branched alkyl;
R⁷ is selected from the group consisting of hydrogen, R⁵OCO, and;
hydrates, solvates, clathrates and acid addition salts thereof; in combination with one or more pharmaceutically acceptable carriers and excipients; pharmaceutical compositions comprising such compounds; or, for the preparation of medicaments for such treatment;
with the proviso that the viral infection is not mediated by Hepatitis C Virus.
